(19) United States
(12) Patent Application Publication
(10) Pub. No.: US 2012/0157865 A1
(43) Pub. Date: Jun. 21, 2012

(54) ADAPTIVE ECG WANDERING CORRECTION

(76) Inventors: Yosef Stein, Sharon, MA (US); Yaniv Sapir, Newton Upper Falls, MA (US)

(21) Appl. No.: 12/973,251

(22) Filed: Dec. 20, 2010

(51) Int. Cl. A61B 5/0408

(52) U.S. Cl. 600/509

(57) ABSTRACT

A received signal is filtered by filtering a group of estimated components of the received signal from the received signal such that the noise and/or distortion in the received signal is minimized.

### FIELD OF THE INVENTION

The invention relates generally to noise reduction in signals.

### BACKGROUND

Surface electrocardiograms (ECGs) have long been central to the diagnosis, management and prognosis of patients with coronary disorders. In most cases, clinical care focuses on physician examination of an ECG and any arrhythmias detected through continuous monitoring.

To obtain ECG waveforms, electrodes are connected to a patient for detecting the ECG signal. Sometimes the signal is provided to a recording device worn by the patient. The sensed signal data are analyzed, periodically or in real-time, for diagnostic purposes. Frequently, the analysis of the ECG signal data becomes erroneous because the baseline of the observed ECG waveform varies considerably. Such variations are known as baseline wander, and can be caused by, e.g., respiration, poor electrode contact and sweating. There are both high and low amplitude wanders across a range of frequencies. Because the spectra of baseline wander and the ECG signal are very close, and in some cases may overlap, it is difficult to eliminate the wander and leave the ECG signal undistorted, especially when real-time monitoring is required.

Some filtering techniques for removing baseline wander from an ECG waveform are known. For example, because the low-frequency components of the wander noise tend to cause the greatest distortion in the ECG signal, some systems simply apply the ECG signal to a high-pass filter, which rejects the low-frequency components. The problem with this approach, however, is that it removes low-frequency components of the ECG signal along with the wander noise; in effect, a new form of distortion is the price of removing the distortion caused by baseline wander. Some methods employ noise-rejection techniques based on wavelet analysis, but such approaches involve highly complex computations that are difficult to implement in real-time systems, and in any case involve significant hardware requirements.

### SUMMARY OF THE INVENTION

In various embodiments of the present invention, efficient, fast, and accurate filtering of the wandering signal (i.e., wandering noise) from an observed ECG signal is provided so that the ECG signal can be analyzed in real time. This is achieved, in part, by estimating several signal components comprising the observed ECG signal, each component corresponding to a distinct frequency in the spectrum of the wandering noise contained in the signal. One or more of the estimated signal components are filtered from the observed ECG signal such that the signal-to-noise and distortion (SINAD) ratio is maximized.

Each estimated signal component includes a desired or true ECG signal component and an unwanted wander noise component at the frequency associated with the estimated signal component. If one estimated signal component is filtered from the observed ECG signal, a portion of the wandering noise together with a portion of the desired ECG signal is also removed. This decreases noise in the ECG signal, but also adds some distortion.

In order to maximize the SINAD of the ECG signal, one or more estimated signal components are selected and filtered such that the portion of the wandering noise removed is maximized, while the portion of the desired ECG signal removed is minimized. To facilitate this, various groups of the estimated signal components are defined. Each group is filtered from the ECG signal to obtain a filtered ECG signal, and using the filtered ECG signals a SINAD value is approximated. The filtered ECG signal corresponding to a maximized SINAD value is selected and used in place of the observed signal for diagnostic or other purposes.

The construction of the various groups of the estimated components of the observed signal and filtering of those groups can be accomplished substantially simultaneously. This enables quick selection of the filtered ECG signal having a maximized SINAD value, and thus facilitates real-time analysis of the ECG signal. As used herein, the term “substantially” generally means±10%, and in some embodiments, ±5%.

Accordingly, in one aspect, embodiments of the invention feature an adaptive system for processing a received signal containing wander noise. The signal to be processed and/or the wander noise includes a plurality of harmonic components. The adaptive system includes a set of comb filters, each comb filter filtering a subset of adaptively estimated harmonic components form the signal. The set of comb filters generates a number of filtered signals, one of which is selected by a decision unit. The decision unit selects one of the filtered signals in response to a metric associated with the filtered signals such that the wander noise associated with the signal is minimized. One or more of the comb filters can be notch filters and/or band-pass filters.

In some embodiments, the adaptive system includes an estimator for estimating the signal components, and the estimator may include a memory buffer and a sliding FFT estimator. The memory buffer stores samples of the received signal and the sliding FFT estimator uses the stored samples to adaptively estimate the amplitudes and/or phases of the components of the received signal. Adaptively means that the estimates are generated in response to the most recently received signal samples, which may represent recent changes in the wander noise.

In some embodiments, the estimator includes a memory buffer, a sliding correlator, a whitening matrix, and a least-square amplitude and phase estimator. The sliding correlator generates a transform matrix used by the least-square estimator corresponding to each sample of the received signal. The whitening matrix may moderate the noise in the transform matrix by making the noise uniform. The least-square estimator uses the moderated transform matrix to adaptively estimate the amplitudes and/or phases of the components of the received signal.

The adaptive system may also include a group generator for generating one or more groups of signal components using estimates of signal components. Each component in a group may correspond to a received-signal component having a certain frequency. The frequencies of the components in a group may be continuous or discontinuous. A group of components having continuous frequencies may be filtered using a band-pass comb filter, and a group of components having discontinuous frequencies may be filtered using a number of notch and/or comb filters. The adaptive system may also include a plurality of energy calculators, each energy calculator calculating an energy value of one of the filtered signals. The metric associated with the plurality of filtered signals is related to the calculated energy values or a rate of change of the energy values.

In some embodiments, the decision unit of the adaptive system selects one of the filtered signals by identifying a group which, when filtered from the received signal, results in a minimum residual wander noise. The decision unit may, additionally, or in the alternative, select one of the filtered signals by identifying a group which, when filtered from the received signal, results in a minimum desired-signal distortion, i.e., the removal of the required signal components along with the removal of wander-noise components.

In another aspect, the invention features a method of filtering a received signal containing a plurality of components such that at least one component contains a wander-noise signal. The method includes filtering, using comb filters, a plurality of groups of signals from the received signal to obtain a plurality of filtered signals. The comb filtering includes band-pass filtering and/or notch filtering in which signals components associated with one or more frequencies are removed from the received signal. The signal components that are removed are obtained from estimates of one or more components of the received signal. The method also includes selecting a filtered signal based on a metric associated with the plurality of filtered signals such that the wander noise associated with the received signal is minimized.

In some embodiments, the method includes estimating components of the received signal for generating the groups of signals that is filtered using comb filtering. The estimation of the signal components may include receiving and storing samples of the received signal, and adaptively estimating an amplitude of each component, by applying sliding FFT to the stored samples.

The estimation of components may also include storing samples of the received signal, receiving a new sample of the received signal, and generating a transform matrix in response to the new sample and the stored samples. The transform matrix is represented in terms of the estimated amplitudes of the sine and cosine components and the dc portion of the samples of the received signal. The noise in the received signal samples is moderated by applying whitening to the generated transform matrix, i.e., by making the noise in the matrix uniform. The amplitudes of sine, cosine, and dc portions of each component of the received signal are estimated based on the whitened matrix.

In some embodiments, the method of filtering includes generating one or more groups of estimated received-signal components, which includes sorting the estimated received-signal components according to a frequency corresponding to each component, and generating a group having an index k by selecting the first k sorted components. The value of k can range from 1 through the number of estimated received-signal components.

The filtering method may also include computing a plurality of energy values, each value corresponding to one filtered signal, such that the metric associated with the plurality of filtered signals is related to the computed energy values. One of the filtered signals is selected in response to the metric associated with the plurality of filtered signals. The metric may be a rate of change of the computed energy values, and the first filtered signal corresponding to which the rate of change of energy is less than a pre-determined threshold value is selected.

In some embodiments the selection includes identifying a group which, when filtered from the received signal, results in a minimum residual wander noise. Additionally, or in the alternative, the selection may include identifying a group which, when filtered from the received signal, results in a minimum desired-signal distortion.

In some embodiments, the filtering method includes generating one or more groups of estimated received-signal components. The groups are generated by selecting a predetermined number of estimated received-signal components according to a selection scheme, and by summing the selected components.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 shows an observed ECG signal;

FIG. 2 shows a filtered ECG signal from which the wandering signal has been removed;

FIG. 3 depicts the ideal values of the signal to noise and distortion (SINAD) ratio obtained by filtering an exemplary ECG signal;

FIG. 4 shows a block diagram of an embodiment of a wandering filter according to the present invention;

FIG. 5 depicts energy values of various filtered ECG signals;

FIGS. 6a and 6b show block diagrams of the sliding fast Fourier transform (FFT) and least-square estimation for estimation of signal components, respectively; and

FIG. 7 shows a flow diagram of an exemplary filtering process.

### DETAILED DESCRIPTION

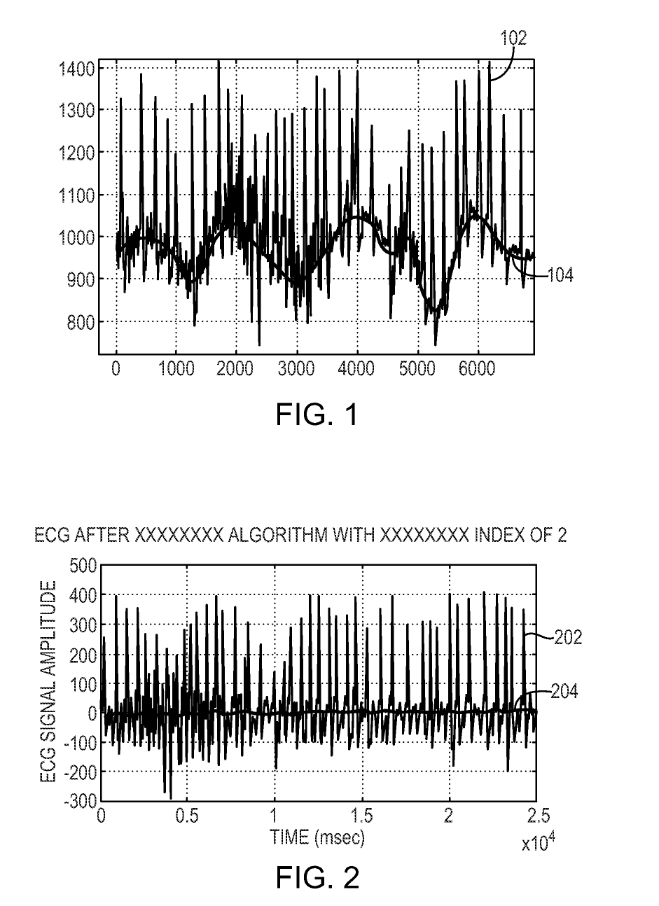

As shown in FIG. 1, in a typical ECG signal 102 recorded from electrodes attached to a patient, the baseline 104 of the signal 102, i.e., a reference voltage relative to which the voltage of the signal 102 varies in time, is not steady (i.e., substantially constant). The baseline 104 wanders, changing over a certain range over time at a certain frequency, thereby adding wandering noise to the true or desired ECG signal contained in the signal 102. The values of the observed ECG signal 102 relative to the baseline 104 are required in subsequent analysis of the ECG signal 102 for diagnostic or other purposes. These relative values are obtained by removing, i.e., filtering the wandering noise. In the filtered ECG signal 202 shown in FIG. 2, the baseline 204 is substantially flat, and does not vary with time. Therefore, the values of the filtered signal 202 can be used directly in diagnosis and treatment of a patient. It should be understood that although various embodiments of the invention are described below with reference to ECG signals, the methods and systems according to the invention can be used to filter any signal containing wandering noise.

An observed ECG signal, denoted as y[n], includes a desired or true ECG signal, denoted as S_{ECG}[n] and wandering noise denoted as W[n]. Then y[n] is given by

Y[n]=S_{ECG}[n]+W[n] (1)

The spectrum of a signal generally includes components of distinct frequencies denoted as f₁, f₂, . . . , f_{K}, where K is the number of distinct frequencies. A component corresponding to a frequency fₖ is described by the amplitude aₖ and phase θₖ of sine and/or cosine waveforms having frequency fₖ. Then, the signal can be approximated as a summation of all components corresponding to frequencies ranging from f₁ through fₖ.

Thus, the wandering signal W[n] of Equation (1) is expressed in term of K components of the wandering-noise spectrum in Equation (2). The S_{ECG}[n] signal includes components corresponding to one or more of the K frequencies, and it typically also includes additional components corresponding to frequencies other than f₁ through f_{K}. Therefore, in Equation (2) S_{ECG}[n] is represented as a portion S¹_{ECG}[n] not represented by the K frequencies, and a portion represented by the summation of components corresponding to the K frequencies of the wandering-noise spectrum. Thus, y[n] of Equation (1) is given by

$$y [ n ] \approx {S}_{ECG}^{1} [ n ] + {\sum }_{k = 1}^{K} {a}_{ECG , k} sin ( {f}_{k} n + {φ}_{k} ) + {\sum }_{k = 1}^{K} {a}_{w , k} sin ( {f}_{k} n + {ϕ}_{k} ) \qquad ( 2 )$$

where a_{ECG,k} and φₖ are the amplitude and phase, respectively, of a component of the desired ECG signal corresponding to the k-th frequency fₖ, and a_{W,k} and φₖ are the amplitude and phase, respectively, of a component of the wandering noise corresponding to the frequency fₖ. The parameters a_{ECG,k}, φₖ, a_{W,k} and φₖ are selected such that the error in approximating y[n] is minimized.

In eliminating wandering noise from the received ECG signal y[n] if the frequency f₁ is filtered out, the noise would decrease by a_{W,1} sin(f₁+φ₁). But, when frequency f₁ is selected for removal, the filter cannot distinguish between a signal component of the wandering noise and that of the desired or true ECG. The filter would simply remove both components, each corresponding to frequency f₁. As a result, a portion of the desired ECG signal, represented by a_{ECG,1} sin(f₁+φ₁) would also be filtered out, thereby introducing a distortion. For simplicity of discussion below, we assume that the K frequencies are arranged in ascending order. Then, by removing from the observed ECG signal the signal components corresponding to the first L frequencies, a portion of the wandering noise is retained. This portion, denoted as Wᵣₑₛ (i.e., the residual wandering noise), is given by

$${W}_{res} [ n ] \approx {\sum }_{k = L + 1}^{K} {a}_{W , k} sin ( {f}_{k} n + {φ}_{k} ) \qquad ( 3 )$$

Similarly, the distortion introduced due to the removal of the desired or true ECG signal components corresponding to the first L frequencies is given by

$${ECG}_{dist} [ n ] = {\sum }_{k = 1}^{L} {a}_{ECG , k} sin ( {f}_{k} n + {φ}_{k} ) \qquad ( 4 )$$

The signal-to-noise and distortion ratio (SINAD) is a ratio of the energy of the desired ECG signal S_{ECG} and the sum of the energy of the residual wandering noise Wᵣₑₛ and the energy of the distortion ECG_{dist}. Thus, the SINAD for a filtered ECG signal is given by

$$SINAD = \frac{{‖ {S}_{ECG} [ n ] ‖}^{2}}{{‖ {ECG}_{dist} [ n ] + {W}_{res} [ n ] ‖}^{2}} \qquad ( 5 )$$

The number of signal components (i.e., L) to be removed from the observed ECG signal can be determined to be Lₒₚₜ such that the SINAD given by Equation (5) is maximized. In other words, the removal of the signal components corresponding to the first Lₒₚₜ frequencies would collectively result in the least amount residual wandering noise and distortion.

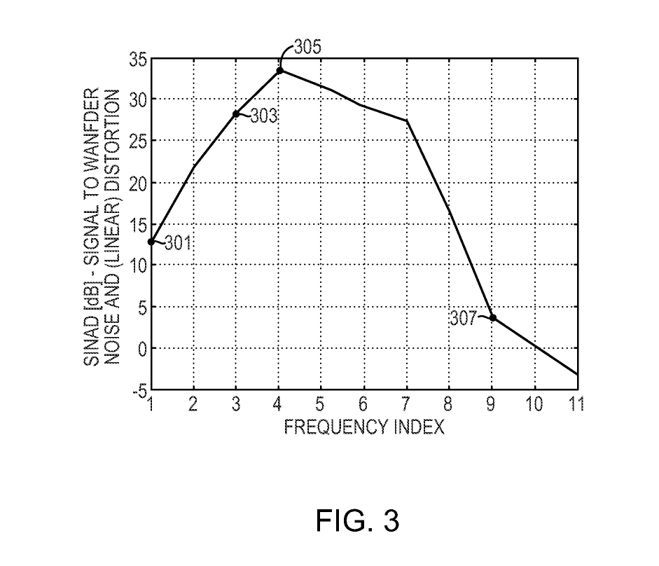

To illustrate, at the data point 301 in FIG. 3, the SINAD is approximately 12 dB. The data point 301 corresponds to the observed, unfiltered ECG signal. Compared to the unfiltered signal, the SINAD increases to approximately 27 dB at data point 303, i.e., when the first two signal components are removed. The maximum SINAD of approximately 33 dB, shown at data point 305, is obtained when the first three signal components are removed. If additional signal components are removed, however, the SINAD decreases. For example, at data point 307, which indicates filtering of the first eight signal components, the approximate SINAD is 4 dB—worse than that of the unfiltered signal. Thus, the Lₒₚₜ for the ECG signal analyzed in FIG. 3 is three, and removing the first three signal components results in the least amount of residual wandering noise and distortion.

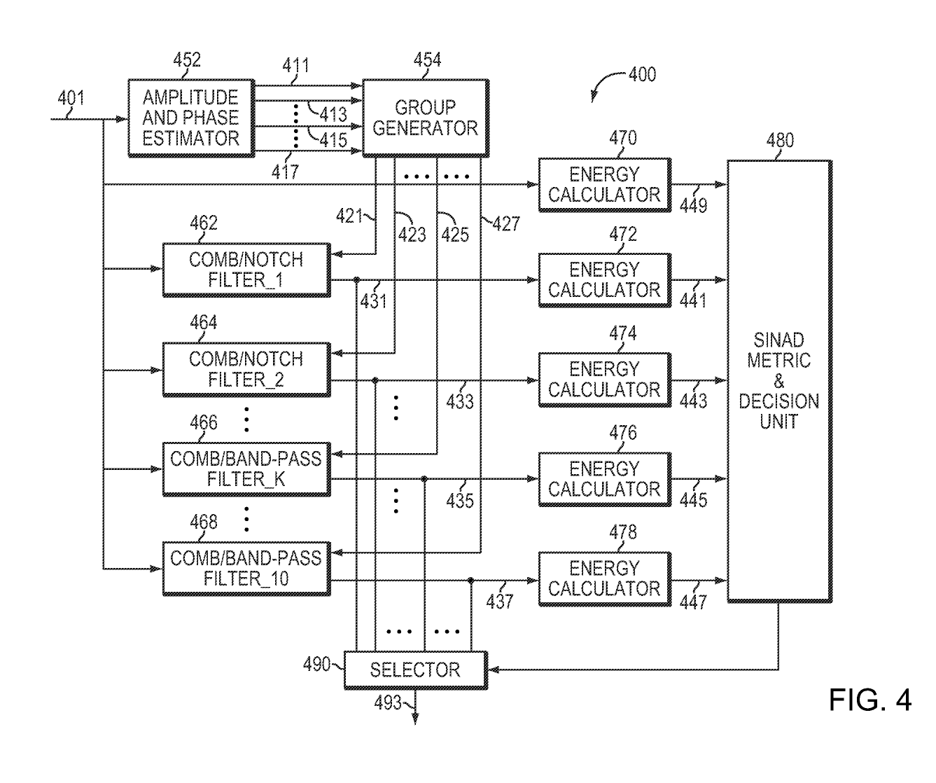

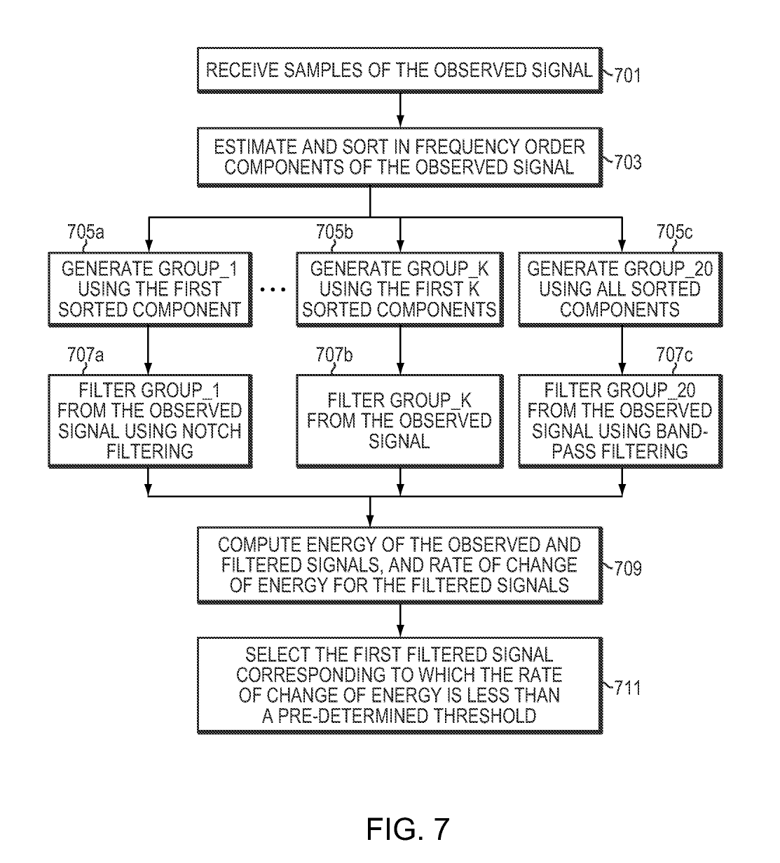

Although the Equations (1)-(5) provide a basis for effectively filtering out the wandering noise from an observed ECG signal, the application of these equations in practice typically requires certain modifications described below with reference to FIGS. 4 and 7. One reason why Equations (1)-(5) cannot be applied directly is that the desired ECG (i.e., S_{ECG}[n]) and the wandering noise (i.e., W[n]) portions of the observed ECG signal y[n] cannot be isolated from the observed ECG signal and analyzed separately. Indeed, filtering out the unwanted wandering noise from the observed ECG signal is one of the objectives the present invention. Therefore, the term ∥S_{ECG}[n]∥², and the parameters a_{ECG,k}, φₖ, a_{W,k} and φₖ cannot be determined individually.

The wandering noise in a typical ECG signal includes components corresponding to several distinct frequencies forming a spectrum. The number of frequencies in a spectrum is denoted as K, and the frequencies are denoted as f₁, f₂, . . . , fₖ, . . . f_{K}, where f₁ is the smallest frequency, fₖ is the k-th frequency, and f_{K} is the largest frequency. Generally in a spectrum, the amplitude of a wandering-noise component varies inversely with the frequency of the component. Thus, the low-frequency wandering-noise components in the spectrum have large amplitudes, and the high-frequency components have small amplitudes.

A desired component of the ECG signal is also associated with each frequency fₖ in the wandering-noise spectrum. The amplitudes of the desired ECG signal components, however, increase with the frequency of the component. In addition, components of the desired ECG signal exist at frequencies not included in the spectrum of the wandering noise. Table 1 shows an exemplary set of 10 frequencies of a wandering-noise spectrum, and the amplitudes of the wandering noise and desired ECG components for each frequency.

TABLE 1

| | | Amplitude of | Amplitude of |
|---|---|---|---|
| | Frequency | wandering noise | desired ECG |
| No. | (Hz) | component (mV) | component (mV) |
| 1 | 0.1 | 0.3 | 0.003 |
| 2 | 0.2 | 0.1 | 0.007 |
| 3 | 0.3 | 0.05 | 0.02 |
| 4 | 0.4 | 0.02 | 0.03 |
| 5 | 0.5 | 0.001 | 0.034 |
| 6 | 0.6 | 0.0007 | 0.035 |
| 7 | 0.7 | 0.0005 | 0.2 |
| 8 | 0.8 | 0.0002 | 0.9 |
| 9 | 0.9 | 0.00005 | 1.0 |
| 10 | 1.0 | 0.00001 | 1.51 |

It should be understood that the spectrum according to Table 1 is illustrative only and that spectrums comprising fewer (i.e., as few as two) and more frequencies, having different ranges of frequencies, and having non-uniform distribution of those frequencies are within the scope of the invention. The amplitudes of the desired ECG and wandering noise components may also have different values.

As shown in FIG. 4, in the filtering system 400 an amplitude and phase estimator 452 receives the observed ECG signal 401 and estimates the combined signal components 411-417. Each signal component corresponds to one of the 10 frequencies (denoted as fₖ) in the spectrum of the wandering noise of the observed ECG signal 401. A combined signal component, as the name suggests, represents both the desired or true ECG component and the wandering noise component corresponding to the frequency fₖ. The components are arranged in ascending order of their frequencies, i.e., the component 411 corresponds to the lowest frequency in the wandering noise spectrum and the component 417 corresponds to the highest frequency in the spectrum. The estimate of each combined signal component is provided in terms of the component's estimated amplitude and phase.

In one embodiment, the group generator 454 generates 10 groups using the combined signal components 411-417. The first group 421 includes the first component 411, i.e., the component corresponding to the lowest frequency (i.e., 0.1 Hz) in the wandering-noise spectrum. The second group 423 is obtained by summing the first two components 411, 413. In general, the k-th group 425 is obtained by summing the combined components corresponding to the k lowest frequencies in the wandering-noise spectrum. The last group 427 includes all 10 combined components.

In another embodiment, the group generator 454 generates 10 (or fewer or more) groups by selecting one combined signal component or summing two or more combined signal components 411-417 selected according to unordered grouping schemes (e.g., random selection). In these grouping schemes the combined signal components are not sorted according to their frequencies.

Notch or comb filter_1 462 receives and filters the first group 421 from the observed ECG signal 401. In the embodiment illustrated using FIG. 4, the first group 421 includes only one combined component corresponding to the frequency f₁. Accordingly, the notch/comb filter_1 462 filters out the frequency f₁. As described above, in other embodiments the first group 421 may include more than one component. For example, the first group 421 may include components corresponding to frequencies f₂, f₄, and f₇, or components corresponding to frequencies f₃ and f₈. In these embodiments, the notch/comb filter 462 filters out frequencies of the components included in the group from the received ECG signal.

Similarly, notch/comb filter_2 464 receives and filters the second group 423 from the ECG signal 401 and, in general, a band-pass/comb filter_k receives and filters the k-th group 425 from the ECG signal 401. If the frequencies of components included in the k-th group are continuous (e.g., from f₁ through fₖ), then filter_k operates as a band-pass filter. Otherwise, filter_k operates as a comb filter that filters out discontinuous frequencies within a range. Each of the filters 462-468 is a digital filter, and although the filtering system 400 includes two notch/comb filters 462, 464 and eight band-pass/comb filters, other configurations comprising fewer or more notch, comb, and band-pass filters, only notch and/or band pass filters, and/or comb filters, and/or other types of digital filters are within the scope of the invention. The filtering substantially removes one or more combined components included in the input group from the observed ECG signal 401. As a result, the energy of each of the filtered signals 431-437 is less than the energy of the observed ECG signal 401.

In the embodiment illustrated in FIG. 4, each successive group among groups 423-427 includes one combined signal component in addition to the components included in the immediately preceding group. Accordingly, the total amount of energy reduced increases with the filtering of each successive group. Due to the above-described characteristics of the ECG signals, however, the incremental amount of energy reduced due to the filtering of a group immediately succeeding a group decreases until a group denoted as Gₒₚₜ is filtered. In other words, until Gₒₚₜ is filtered, the rate of reduction of energy of the filtered signal decreases with the filtering of successive groups. This phenomenon occurs because until Gₒₚₜ is filtered, the additional amount of wandering noise removed by filtering each group, which is proportional to the amplitude of the additional wandering noise component in that group, generally decreases with the filtering of successive groups.

In the groups following Gₒₚₜ, the amplitudes of the wandering noise components are not substantial. As a result, decrease in the filtered signal energy due to the removal of wandering noise is not substantial. However, the amplitudes of the desired ECG signal components in these groups are substantial. Moreover, these amplitudes generally increase with each successive group. Therefore, the total reduction in the filtered signal energy due to filtering of the successive groups after Gₒₚₜ increases. Importantly, the incremental reduction in the filtered signal energy (i.e., the rate of energy reduction) increases because the amount of energy reduced due to the removal of the desired ECG components increases.

Therefore, Gₒₚₜ can be determined by computing the rate of reduction of energy of the filtered signals 431-437. A condition at which the rate of reduction of energy has reached a lowest value, and beyond which the rate increases, identifies Gₒₚₜ. It should be understood, however, that the rate of reduction of energy is only an illustrative metric. In other embodiments, such as those using unordered grouping schemes, other metrics (e.g., median, average, etc.) may be used to determine Gₒₚₜ.

By filtering the combined signal components in Gₒₚₜ wandering noise is maximally eliminated while distortion is minimized by minimizing the number of desired or true ECG signal components that are removed. Therefore, the energy of the filtered signal due to filtering Gₒₚₜ represents the maximized SINAD given by Equation (5). Accordingly, the filtered ECG signal output by the filter having Gₒₚₜ as an input is the desired ECG signal having minimized wandering noise and distortion.

The SINAD metric and decision unit 480 receives energy values 441-447 from energy calculators 472-478 that calculate energies of filtered signals 431-437, respectively. Typically, the energy calculator 472 calculates the signal energy by squaring the amplitudes of a pre-determined number (e.g., N) of samples of the filtered signal 431, and by summing the amplitude squares. The SINAD metric and decision unit 480 also receives energy 449 of the unfiltered, observed ECG signal 401 from the energy calculator 470. Using these energy values, the SINAD metric and decision unit 480 computes the rate of reduction of energy as the successive groups 421-427 are filtered, and identifies Gₒₚₜ as described above. The SINAD metric and decision unit 480 may also be configured to identify Gₒₚₜ according to other suitable metrics as described above.

The SINAD metric and decision unit 480 provides an index signal 483 representing the index of Gₒₚₜ (e.g., 3, 4, etc.) to the selector 490. The selector 490 receives the filtered signals 431-437 and selects the filtered signal obtained by filtering Gₒₚₜ using the index signal 483. The selected filtered signal is output as the desired ECG signal 493 that may be used in diagnosis and/or treatment.

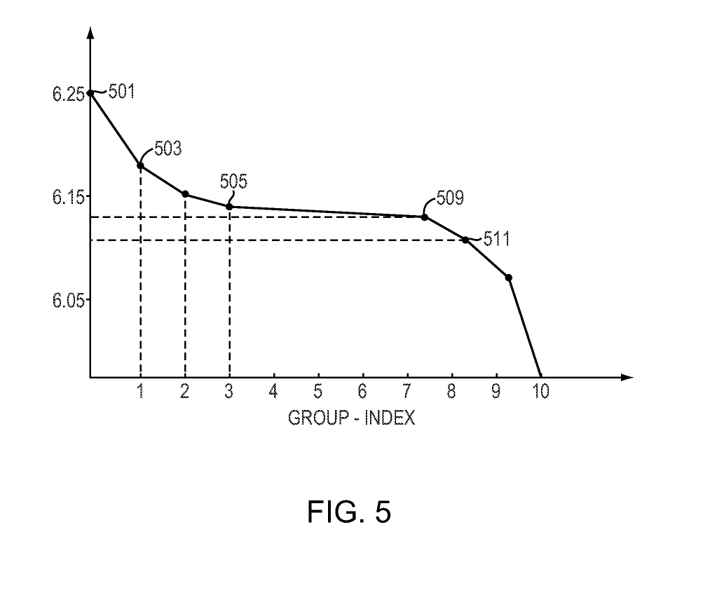

The operation of the SINAD metric and decision unit 480 according to one embodiment is demonstrated with reference to FIG. 5. The data point 501 corresponds to the unfiltered ECG signal 401, and shows that the energy of the ECG signal 401 is approximately 6.25 mJ. The energy of the first filtered signal 431 is approximately 6.17 mJ as shown at data point 503, indicating a reduction of approximately 0.08 mJ in the energy of the ECG signal 401. However, at the data point 505, which shows the energy of the signal obtained by filtering the third group as approximately 6.14 mJ, the incremental reduction compared to the data point 503 is only 0.03 mJ. The data point 509, which corresponds to the signal obtained by filtering group 7, shows that the incremental reduction in energy relative to data point 505 is substantially zero.

The data point 511 corresponds to the signal obtained by filtering group 8. The energy of the filtered signal represented by the data point 511 is approximately 6.06, indicating a substantial reduction in energy of approximately 0.07 mJ relative to data point 509. As described above, the SINAD metric and decision unit 480 calculates the incremental reduction in the energy of the filtered signals 431-437 and determines the index of Gₒₚₜ as 3.

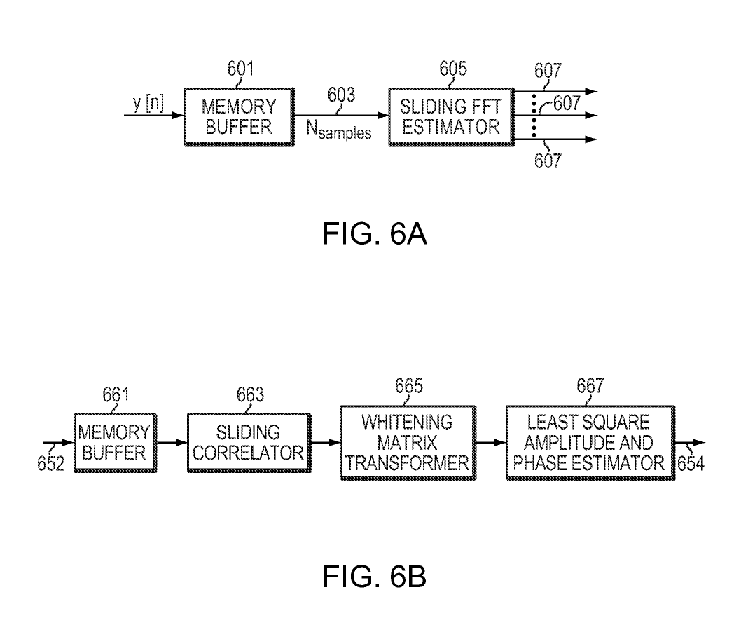

The filtering of an ECG signal as described above requires determining the amplitudes and phases of the combined signal components corresponding to the frequencies in the wandering-noise spectrum. Two methods of computing the component amplitudes, namely, sliding fast Fourier transform (FFT) and least-square estimation, are described with reference to FIGS. 6a and 6b, respectively. Other methods of amplitudes and phase estimation, such as the conventional FFT, sliding-window least square, etc., are within the scope of the invention.

The memory buffer 601 receives samples of the observed ECG signal y[n]. Corresponding to the m-th received sample, the buffer 601 stores a total of N subsequently received samples, denoted as yₘ, yₘ₊₁, . . . , y_{m+N-1}. In general, larger the number of samples used in estimating the amplitudes and phases of signal components, more accurate are the amplitude and phase estimates. As more samples are used, however, the estimation requires more computations, and therefore additional circuitry and time for the estimation. Accordingly, the number of samples N is selected such that the estimation circuitry is not too large and/or slow while yielding the required level of accuracy. In one embodiment, the number of samples is equal to the number of distinct frequencies (i.e., 10) in the spectrum of the wandering noise.

Using the conventional FFT, the amplitude of a combined signal component corresponding to the k-th frequency in the wandering-noise spectrum for the m-th received sample of the observed ECG signal is given by

$${a}_{k}^{m} = {\sum }_{n = 0}^{N - 1} {y}_{m + n} {e}^{- j \frac{2 π}{N} kn} \qquad ( 6 )$$

According to Equation (6), for each new sample received, and for each frequency k, N products and N−1 additions must be performed. The sliding FFT estimator 605 avoids having to perform these computations by taking advantage of the fact that the set of N samples corresponding to the (m+1)-th received ECG signal sample retains N−1 samples from the previous set (i.e., samples yₘ₊₁, yₘ₊₂, . . . , y_{m+N-1}). One new sample, denoted as y_{m+N} is added to the previous set and one old sample, yₘ, is removed from the previous set to obtain the set corresponding to the received sample yₘ₊₁.

As a result, the amplitude estimates corresponding to each of the k frequencies for the (m+1)-th sample of the observed ECG signal, denoted as aₖ^{m+1} can be obtained using the previously computed estimates aₖ^{m} as

$${a}_{k}^{m + 1} = [ {a}_{k}^{m} - {y}_{m} ] {e}^{j \frac{2 π}{N} k} + {y}_{m + N} {e}^{- j \frac{2 π}{N} k ( N - 1 )} \qquad ( 7 )$$

According to Equation (7), the computation of aₖ^{m+1} requires only one subtraction, two multiplications, and two additions as opposed to requiring N multiplications and N−1 additions as required by Equation (6). As N is typically larger than 2 (e.g., 10, 20, etc.) the circuitry required to execute Equation (7) is smaller compared to that required to execute Equation (6). The sliding FFT estimator 605 receives N samples 603 from the memory buffer 601 and performs the computations required according to Equation (7) and estimates amplitudes of the combined signal components 607 for every received sample of the observed ECG signal. These estimated amplitudes are supplied to the group generator 454 and used in filtering the observed ECG signal as described above with reference to FIG. 4.

When the spectrum of the wandering noise includes non-orthogonal (i.e., mutually correlated) frequencies, the amplitudes and phases of the combined signal components are determined using the least-square estimation method. The (m+n)-th sample of the observed ECG signal can be written as

$${y}_{n + m} = {a}_{0}^{m} + {\sum }_{k = 1}^{K} {a}_{k}^{m} cos ( {f}_{k} ( n + m ) ) + {\sum }_{k = 1}^{K} {b}_{k}^{m} sin ( {f}_{k} ( n + m ) ) \qquad ( 8 )$$

where a₀^{m} is the estimated DC component of the ECG signal. A combined signal component corresponding to the k-th frequency includes sine and cosine waveforms. The coefficients aₖ^{m} and bₖ^{m} represent the amplitudes of the sine and cosine waveforms, respectively, corresponding to the k-th frequency for the m-th sample.

Using Equation (8), N samples of the ECG signal are given by

$$\begin{matrix}[ \begin{matrix}{y}_{m} \\ {y}_{1 + m} \\ ⋮ \\ ⋮ \\ {y}_{N - 1 + m}\end{matrix} ] = {H}_{m} [ \begin{matrix}{a}_{0}^{m} \\ {a}_{1}^{m} \\ {a}_{2}^{m} \\ ⋮ \\ {a}_{k}^{m} \\ {b}_{1}^{m} \\ ⋮ \\ {b}_{k}^{m}\end{matrix} ] where & ( 9 ) \\ {H}_{m} = [ \begin{matrix}1 & cos ( {f}_{1} m ) & cos ( {f}_{2} m ) & … & cos ( {f}_{k} m ) & sin ( {f}_{1} m ) & … & sin ( {f}_{k} m ) \\ 1 & cos ( {f}_{1} ( m + 1 ) ) & cos ( {f}_{2} ( m + 1 ) & … & cos ( {f}_{k} ( m + 1 ) ) & sin ( {f}_{1} ( m + 1 ) ) & … & sin ( {f}_{k} ( m + 1 ) ) \\ & & & & & & & \\ & & & & & & & \\ ⋮ & ⋮ & ⋮ & ⋮ & ⋮ & ⋮ & ⋮ & ⋮ \\ & & & & & & & \\ & & & & & & & \\ 1 & cos ( {f}_{1} ( m + N - 1 ) ) & & & & & & sin ( {f}_{k} ( m + N - 1 ) )\end{matrix} ] & ( 10 )\end{matrix}$$

Using least-square approximation, the coefficients a₀^{m}, aₖ^{m}, and bₖ^{m} are estimated from the N samples of the observed ECG signal as

$$[ \begin{matrix}{\hat{a}}_{0}^{m} \\ {\hat{a}}_{1}^{m} \\ {\hat{a}}_{2}^{m} \\ ⋮ \\ {\hat{a}}_{k}^{m} \\ {\hat{b}}_{1}^{m} \\ ⋮ \\ {\hat{b}}_{k}^{m}\end{matrix} ] = {[ {H}_{m}^{T} {H}_{m} ]}^{- 1} {H}_{m}^{T} [ \begin{matrix}{y}_{m} \\ {y}_{1 + m} \\ ⋮ \\ ⋮ \\ {y}_{N - 1 + m}\end{matrix} ] \qquad ( 11 )$$

In one embodiment, the matrix [Hₘ^{T}Hₘ]⁻¹Hₘ^{T} must be computed for each newly received sample of the observed ECG signal. The memory buffer 661 receives the observed ECG signal 652 and stores the m-th sample and the N−1 subsequent samples of the ECG signal 652. For each new sample, the sliding correlator 663 generates the matrix Hₘ by deleting the first row of the previous matrix (i.e., matrix Hₘ₋₁), shifting each row of Hₘ₋₁ up by one place, and by replacing the last row with the values [1 cos(f₁(m+N) . . . cos(fₖ(m+N) . . . sin(fₖ(m+N)]. The whitening matrix transformer 665 whitens, i.e., substantially orthogonalizes the matrix Hₘ. The orthogonalization, which reduces the correlation between the cosine and sine components corresponding to different frequencies, increases the accuracy of the estimates obtained using the transformed matrix Hₘ. The least-square amplitude and phase estimator 667 computes [Hₘ^{T}Hₘ]⁻¹Hₘ^{T} using the transformed matrix Hₘ supplied by the whitening matrix transformer 665, and estimates the coefficients â₀^{m}, âₖ^{m}, and \hat{b}ₖ^{m} 654 according to Equation (11). These estimated amplitudes are supplied to the group generator 454 of FIG. 4 and are used in filtering the observed ECG signal as described with reference to FIGS. 4 and 7.

In the exemplary filtering process depicted in FIG. 7, samples of the observed signal are received in step 701. Using these samples, estimates of the amplitudes and phases of components of the observed signal are generated in step 703. Each component is associated with one frequency in the spectrum of the wandering noise contained in the observed signal. The components are sorted in the ascending order of the frequencies associated with the components. The components may also be sorted in the descending order of frequencies.

In step 705a, group_1 is generated by selecting the first sorted component. Similarly, group_2 is generated by selecting the first two sorted components. In general, as shown in step 705b, group_k is generated by selecting the first k sorted components. The last group, i.e., group_20 is generated in step 705c by selecting all 20 components. Although the filtering process 700 shows as steps 705a-c as executing concurrently, it should be understood that this is for illustration only and that in other embodiments some or all steps may be executed sequentially.

Group_1 is filtered from the observed signal using notch filtering in step 707a, and group_20 is filtered from the observed signal using band-pass filtering in step 707c. In general, the k-th group is filtered from the observed signal producing a filtered signal corresponding to group_k, as shown in step 707b. Some or all of the filtering steps 707a-c may be executed sequentially. In step 709, energy of the observed signal is computed. Energy of each of the filtered signals produced in steps 707a-c is also computed. The rate of change of energy of the filtered signals is also analyzed in step 709. Finally in step 711, the rate of change of energy is compared to a pre-determined threshold value. The first filtered signal corresponding to which the rate of change of energy is less than the threshold is selected as the desired filtered signal.

Although the present invention has been described with reference to specific details, it is not intended that such details should be regarded as limitations upon the scope of the invention, except as and to the extent that they are included in the accompanying claims.

What is claimed is:

1. An adaptive system for processing a received signal containing wander noise, and including a plurality of harmonic components, the system comprising:
a set of comb filters, each comb filter filtering a subset of adaptively estimated harmonic components form the signal, the set of comb filters generating a plurality of filtered signals; and
a decision unit for selecting one of the filtered signals from the plurality of filtered signals in response to a metric associated therewith, whereby wander noise associated with the signal is minimized.

2. The system of claim 1, wherein at least one of the plurality of the comb filters is a notch filter.

3. The system of claim 1, wherein at least one of the plurality of the comb filters is a band-pass filter.

4. The system of claim 1, further comprising an estimator for estimating the signal components.

5. The system of claim 4, wherein the estimator comprises:
a memory buffer; and
a sliding FFT estimator.

6. The system of claim 4, wherein the estimator comprises:
a memory buffer;
a sliding correlator;
a whitening matrix; and
a least-square amplitude and phase estimator.

7. The system of claim 1, further comprising a group generator for generating one or more groups of signal components using estimates of signal components.

8. The system of claim 1, further comprising a plurality of energy calculators, each energy calculator calculating an energy value of one of the filtered signals, wherein the metric associated with the plurality of filtered signals is associated with the calculated energy values.

9. The system of claim 1, wherein the decision unit selects one of the filtered signals by identifying a group which, when filtered from the received signal, results in a minimum residual wander noise.

10. The system of claim 1, wherein the decision unit selects one of the filtered signals by identifying a group which, when filtered from the received signal, results in a minimum desired-signal distortion.

11. A method of filtering a received signal containing a plurality of components, at least one component containing a wander-noise signal, the method comprising the steps of:
filtering, using a comb filter, a plurality of groups of signals from the received signal to obtain a plurality of filtered signals, wherein (i) the filtering step comprises at least one of band-pass filtering and notch filtering, and (ii) each group of signals comprising signals obtained from estimates of one or more components of the received signal; and
selecting a filtered signal based on a metric associated with the plurality of filtered signals whereby wander noise associated with the received signal is minimized.

12. The method of claim 11, further comprising the step of estimating components of the received signal for generating the groups of signals.

13. The method of claim 12, wherein the estimating step comprises:
receiving and storing samples of the received signal; and
estimating an amplitude of each component, by applying sliding FFT to the stored samples.

14. The method of claim 12, wherein the estimation step comprises:
storing samples of the received signal;
receiving a new sample of the received signal;
generating a matrix in response to the new sample and the stored samples;
applying whitening to the generated matrix; and
estimating amplitudes of sine, cosine, and dc portions of each component of the received signal based on the whitened matrix.

15. The method of claim 11, further comprising the step of generating one or more groups of estimated received-signal components, the generating step comprising:
sorting the estimated received-signal components according to a frequency corresponding to each component; and
generating a group having an index k by selecting the first k sorted components, where k ranges from 1 through the number of estimated received-signal components.

16. The method of claim 11, further comprising computing a plurality of energy values, each value corresponding to one filtered signal, wherein the metric associated with the plurality of filtered signals is related to the computed energy values.

17. The method of claim 16, wherein (i) the metric is a rate of change of the computed energy values, and (ii) the selecting step comprises selecting the first filtered signal corresponding to which the rate of change of energy is less than a pre-determined threshold value.

18. The method of claim 11, wherein the selecting step comprises identifying a group which, when filtered from the received signal, results in a minimum residual wander noise.

19. The method of claim 11, wherein the selecting step comprises identifying a group which, when filtered from the received signal, results in a minimum desired-signal distortion.

20. The method of claim 11, further comprising the step of generating one or more groups of estimated received-signal components, wherein generating a group comprises:
selecting a predetermined number of estimated received-signal components according to a selection scheme; and
summing the selected components.
